(19) United States
(12) Patent Application Publication
(10) Pub. No.: US 2025/0024014 A1
(43) Pub. Date: Jan. 16, 2025

(54) IMAGE DISPLAY SYSTEM, DISPLAY CONTROL DEVICE, AND IMAGE DISPLAY METHOD

(71) Applicant: OLYMPUS MEDICAL SYSTEMS CORP., Tokyo (JP)

(72) Inventor: Toru KUCHIMARU, Hachioji-shi (JP)

(73) Assignee: OLYMPUS MEDICAL SYSTEMS CORP., Tokyo (JP)

(21) Appl. No.: 18/901,439

(22) Filed: Sep. 30, 2024

Related U.S. Application Data
(63) Continuation of application No. PCT/JP2022/016995, filed on Apr. 1, 2022.

(51) Int. Cl. H04N 13/398; A61B 1/00; H04N 13/207; H04N 13/341; H04N 13/366; H04N 23/50

(52) U.S. Cl. CPC H04N 13/398; A61B 1/00006; A61B 1/0005; H04N 13/207; H04N 13/341; H04N 13/366; H04N 23/555; H04N 2213/008

(57) ABSTRACT

An image display system includes: a monitor configured to be able to display a 3D image; a detection device configured to detect a position of an observer or a position of monitor observation glasses with respect to the monitor as an observation position; and a display control device configured to determine at least one of a display size and a display position of the 3D image to be displayed on the monitor on the basis of the observation position detected by the detection device.

The present invention relates to an image display system, a display control device, and an image display method. This application is a continuation application based on International Patent Application No. PCT/JP2022/016995 filed on Apr. 1, 2022, and the content of the PCT international application is incorporated herein by reference.

### TECHNICAL FIELD

### Background Art

Conventionally, 3D monitors (3D displays) that can display 3D images are used. As 3D monitors, a glasses-type monitor using glasses such as polarized glasses, a naked eye-type monitor not using glasses, and the like have been devised. Since a 3D monitor uses binocular parallax, a range in which a 3D image displayed on the 3D monitor can be appropriately observed (a 3D observable area) is limited to a recommended range defined for each 3D monitor.

In Patent Document 1, a display system including a display means displaying a parallax image including a left-eye image and a right-eye image and a guide means guiding a viewer to a viewing position of a display device is described. The guide means described in Patent Document 1 can perform guidance by displaying a viewing position that is appropriate for stereoscopic vision.

In Patent Document 2, a display system detecting a position and an inclination of stereoscopic glasses with respect to a screen and correcting a left-eye image and a right-eye image is described.

### CITATION LIST

### Patent Document

### [Patent Document 1]

Japanese Unexamined Patent Application, First Publication No. 2012-195728

### [Patent Document 2]

Japanese Unexamined Patent Application, First Publication No. 2011-071898

### SUMMARY OF INVENTION

### Technical Problem

However, in the display systems described in Patent Document 1 and Patent Document 2, a state in which a position of an observer is not included in a range in which a 3D image displayed on a 3D monitor can be appropriately observed (a 3D observable area) may occur. In this case, it is necessary to change the position of the observer with respect to the 3D monitor and correct a left-eye image and a right-eye image.

On the basis of the situations described above, an object of the present invention is to provide an image display system, a display control device, and an image display method capable of displaying an appropriate 3D image for an observer without changing the position of the observer with respect to a 3D monitor and performing extensive correction of images.

### Solution to Problem

In order to solve the problems described above, the present invention proposes the following means.

According to a first aspect of the present invention, there is provided an image display system including: a monitor configured to be able to display a 3D image; a detection device configured to detect a position of an observer or a position of monitor observation glasses with respect to the monitor as an observation position; and a display control device configured to determine at least one of a display size and a display position of the 3D image to be displayed on the monitor on the basis of the observation position detected by the detection device.

According to a second aspect of the present invention, there is provided a display control device that determines at least one of a display size and a display position of a 3D image to be displayed on a monitor on the basis of an observation position that is a position of an observer or a position of monitor observation glasses with respect to a monitor that is able to display the 3D image.

According to a third aspect of the present invention, there is provided an image display method including: a process of detecting a position of an observer or a position of monitor observation glasses with respect to a monitor as an observation position; and a process of determining at least one of a display size and a display position of a 3D image to be displayed on the monitor on the basis of the detected observation position.

### Advantageous Effects of Invention

According to an image display system, a display control device, and an image display method of the present invention, an appropriate 3D image can be displayed to an observer without changing the position of the observer with respect to a 3D monitor and performing extensive correction of images.

### BRIEF DESCRIPTION OF DRAWINGS

FIG. 1 is a diagram illustrating an endoscope system according to a first embodiment.

FIG. 2 is a diagram illustrating an observation position of an observer with respect to a 3D monitor of the endoscope system seen from above.

FIG. 3 is a diagram illustrating an observation position of an observer with respect to the 3D monitor of the endoscope system seen in a horizontal direction.

FIG. 4 is a diagram illustrating a 3D image displayed on the 3D monitor.

FIG. 5 is a diagram explaining an operation of a display control device of the endoscope system.

FIG. 6 is a diagram describing the operation of the display control device.

FIG. 7 is a diagram describing the operation of the display control device.

FIG. 8 is a diagram describing the operation of the display control device.

FIG. 9 is a diagram describing the operation of the display control device.

FIG. 10 is a diagram describing the operation of the display control device.

FIG. 11 is a diagram describing the operation of the display control device.

### DESCRIPTION OF EMBODIMENTS

### First Embodiment

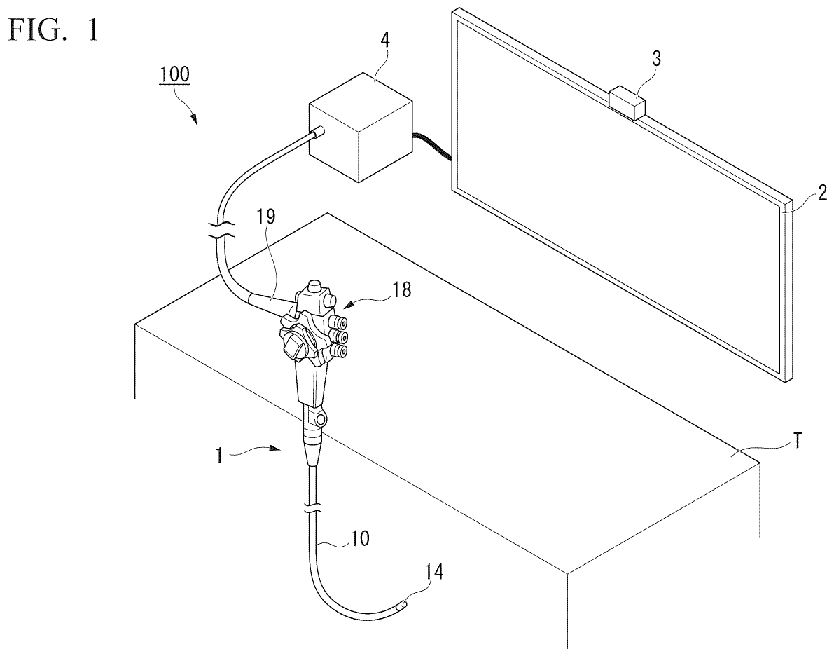

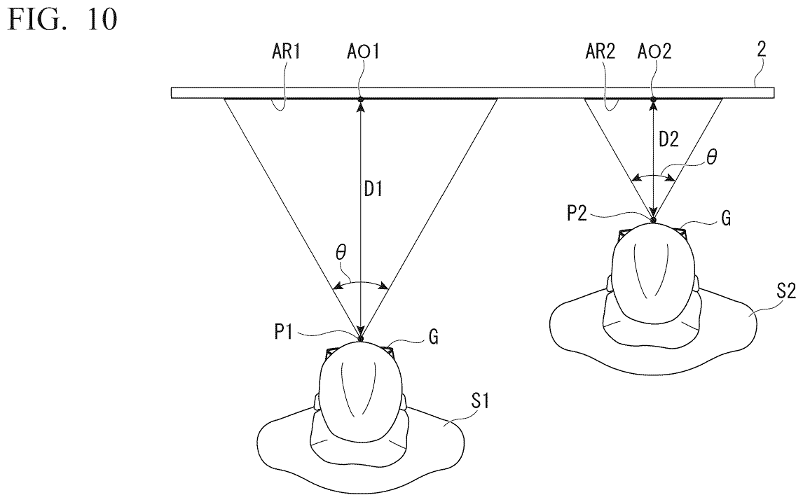

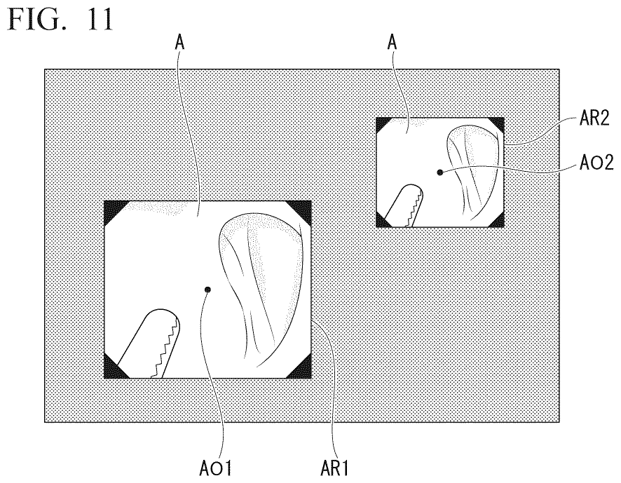

An endoscope system 100 according to a first embodiment of the present invention will be described with reference to FIGS. 1 to 11.

### [Endoscope System 100]

FIG. 1 is a diagram illustrating the endoscope system 100.

The endoscope system (an image display system) 100 includes an endoscope 1, a 3D monitor 2, a detection device 3, and a display control device 4.

### [Endoscope 1]

The endoscope 1 is a device that is used for performing observation and treatment inside of the body of a patient, for example, lying on an operating table T. The endoscope 1 includes an elongated insertion part 10 that is inserted into a patient's body, an operation part 18 connected to a proximal end of the insertion part 10, and a universal cord 19 extending from the operation part 18.

The insertion part 10 has an imaging unit 14 at its tip end. The imaging unit 14 has a stereo optical system and an imaging element such as a CMOS image sensor or the like. The imaging unit 14 can acquire an image capture signal that can be used for generating a stereo image (a 3D image).

The operation part 18 receives an operation on the endoscope 1. The universal cord 19 connects the endoscope 1 and the display control device 4. The imaging unit 14 transmits an image capture signal to the display control device 4 via the universal cord 19.

### [3D Monitor 2]

The 3D monitor (a 3D display) 2 displays an image generated by the endoscope 1 and various kinds of information relating to the endoscope system 100, and the like. The 3D monitor 2 is a monitor that can display a 3D image and, for example, is a glasses-type monitor or a naked eye-type monitor.

The glasses-type monitor is a monitor allowing an observer S to wear glasses having special optical characteristics and displaying images with binocular parallax to both the eyes. The glasses that the observer S observing the glasses-type monitor is wearing are polarized glasses G, liquid crystal shutter glasses, or the like.

The naked eye-type monitor is a monitor that allows an observer S to be able to observe a naked-eye stereoscopic image without using glasses. The naked eye-type monitor is a monitor that can be used for observing a naked-eye stereoscopic image, for example, using a parallax barrier system or a lenticular lens system.

In this embodiment, the 3D monitor 2 is a glasses-type monitor for which an observer S uses polarized glasses G. In addition, the 3D monitor 2 may be a naked eye-type monitor allowing an observer S to be able to observe a naked eye stereoscopic image without using glasses.

### [Detection Device 3]

The detection device 3, for example, is installed in an upper part of the 3D monitor 2 and is a position sensor that detects a position of an observer S with respect to the 3D monitor 2 as an “observation position P”. The position of the observer S with respect to the 3D monitor 2 that has been detected by the detection device 3 is acquired by the display control device 4. The detection device 3, for example, detects polarized glasses G worn by an observer S, a face of the observer S, and the like from a captured image, thereby detecting the position of the observer S with respect to the 3D monitor 2. In addition, the display control device 4 may perform image processing of detecting the polarized glasses G and the face of the observer S from an image captured by the detection device 3.

The detection device 3 may be a position sensor detecting the position of the observer S with respect to the 3D monitor 2, for example, using a laser or infrared rays. In addition, the detection device 3 may be a position sensor detecting a marker or the like embedded in the polarized glasses G.

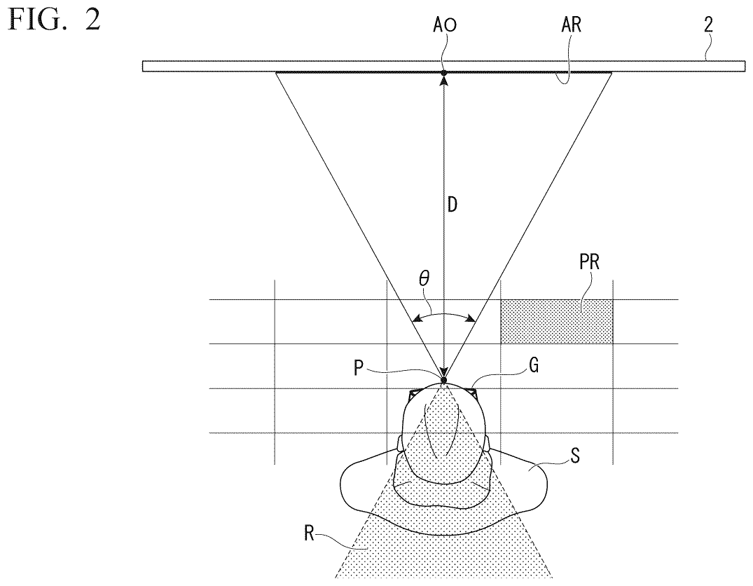

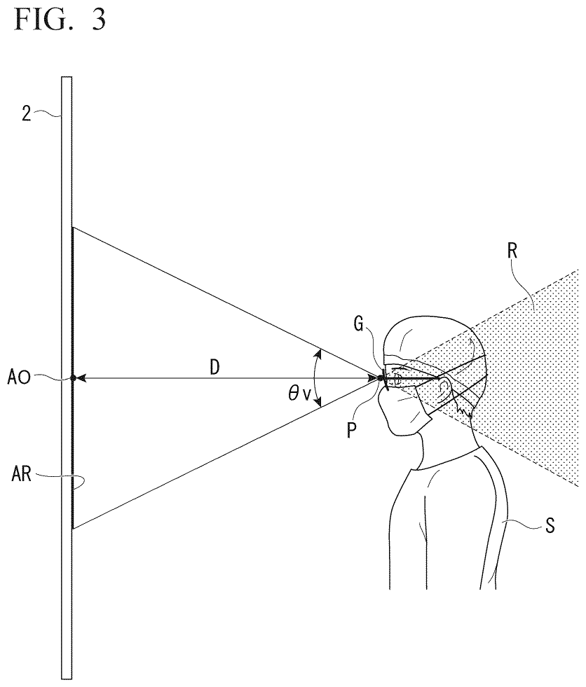

FIG. 2 is a diagram illustrating an observation position P of an observer S with respect to the 3D monitor 2 seen from above. FIG. 3 is a diagram illustrating an observation position P of an observer S with respect to the 3D monitor 2 seen in a horizontal direction. The observation position P, for example, as illustrated in FIG. 2, is a position near both eyes of the observer S. The detection device 3 may set a position located slightly to a 3D monitor 2 side from both the eyes of the observer S as the observation position P.

### [Display Control Device 4]

The display control device 4 is a device that generates a 3D image A on the basis of an image capture signal acquired from the imaging unit 14 and displays the generated 3D image A on the 3D monitor 2. The display control device 4 is a processing circuit (computer) that has a processor such as a CPU, a memory that can read a program, and the like and can execute a program, logic circuits mounted in an ASIC or an FPGA, or a combination thereof.

The display control device 4 may be an arithmetic operation device that is disposed on a cloud server that is connected to the endoscope 1 and the 3D monitor 2 via the Internet.

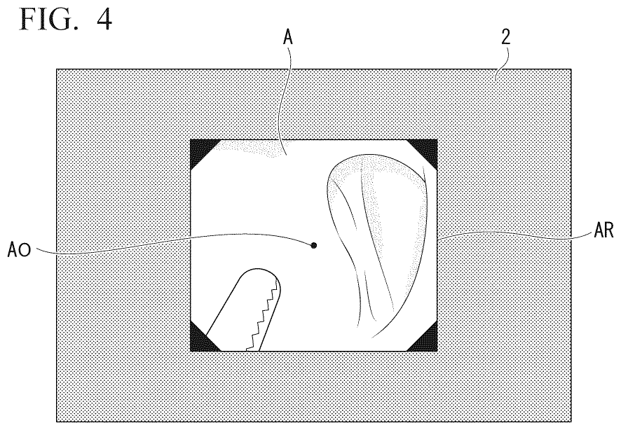

FIG. 4 is a diagram illustrating a 3D image A displayed on the 3D monitor 2.

The display control device 4 determines at least one of a display size and a display position of a 3D image A displayed on the 3D monitor 2 on the basis of the observation position P of the observer S that has been detected by the detection device 3. More specifically, the display control device 4 determines at least one of a display size and a display position of a 3D image A displayed on the 3D monitor 2 such that the observation position P is disposed in a range in which the observer S can appropriately observe the 3D image on the 3D monitor 2 (hereinafter, also referred to as a “3D observable area R”).

In this embodiment, as illustrated in FIG. 2, the display control device 4 determines at least one of a display size and a display position of a 3D image A displayed on the 3D monitor 2 such that the observation position P is disposed closest to 3D monitor 2 in the 3D observable area R.

The display control device 4 determines the display position of the 3D image A displayed on the 3D monitor 2 on the basis of the observation position P from the 3D monitor 2. More specifically, as illustrated in FIG. 2, the display control device 4 sets an intersection of a perpendicular line from the observation position P to the 3D monitor 2 and the 3D monitor 2 as a center AO of a display area AR of the 3D image A.

The display control device 4 determines the display size of the 3D image A displayed on the 3D monitor 2 on the basis of the observation position P from the 3D monitor 2. More specifically, the display control device 4 determines the display size (the size of the display area AR) of the 3D image A on the basis of an observation distance D from the 3D monitor 2 to the observation position P and a viewing angle θ of the 3D monitor 2.

Here, in the 3D monitor 2, a viewing angle θ with which the 3D image A can be optimally observed in accordance with the characteristics of the 3D monitor 2 is defined. More specifically, the viewing angle θ is a vertical viewing angle and a horizontal viewing angle defined in accordance with the liquid crystal size of the 3D monitor 2 and specifications of a polarizing film.

In the 3D monitor 2, for example, right-eye pixels and left-eye pixels are alternately aligned in a vertical direction. In accordance with the arrangement pitch of right-eye pixels and left-eye pixels, a distance between the pixels and the polarizing film, the specifications of the polarizing film, and the like, a light ray angle from the right-eye pixels and the left-eye pixels is determined as a vertical viewing angle θv illustrated in FIG. 3. A horizontal viewing angle illustrated in FIG. 2 is determined also on the basis of the liquid crystal size, the pixel pitch, the specifications of the polarizing film, and the like.

The display control device 4 displays a background color, for example, such as black in an area other than the display area AR on the 3D monitor 2. The display control device 4 may display a GUI image displaying information of the endoscope system 100 in an area other than the display area AR in the 3D monitor 2.

### [Operation of Endoscope System 100]

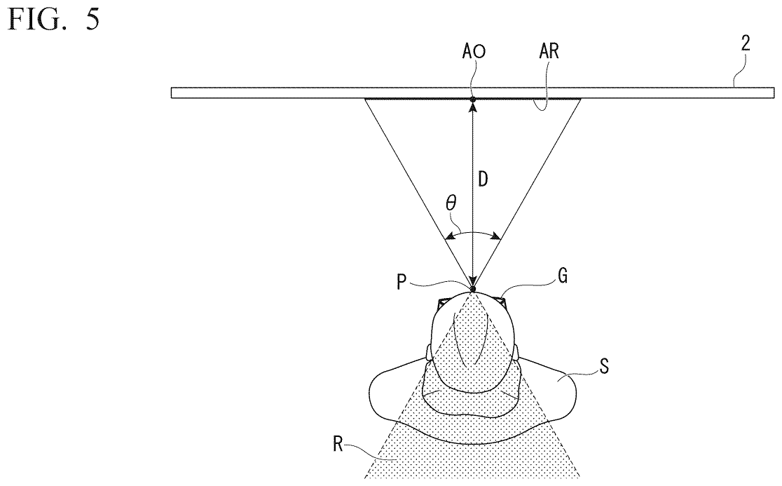

Next, an operation (an image control method) of the display control device 4 of the endoscope system 100 will be described. Here, an operation example of the display control device 4 performed when an observer S has moved will be illustrated. FIGS. 5 to 11 are diagrams describing the operation of the display control device 4.

### <Approach Operation>

When an observer S moves to a position illustrated in FIG. 5 from the position illustrated in FIG. 2, the display control device 4 determines at least one of a display size and a display position of a 3D image A displayed on the 3D monitor 2 on the basis of a new observation position P of an observer S that has been detected by the detection device 3.

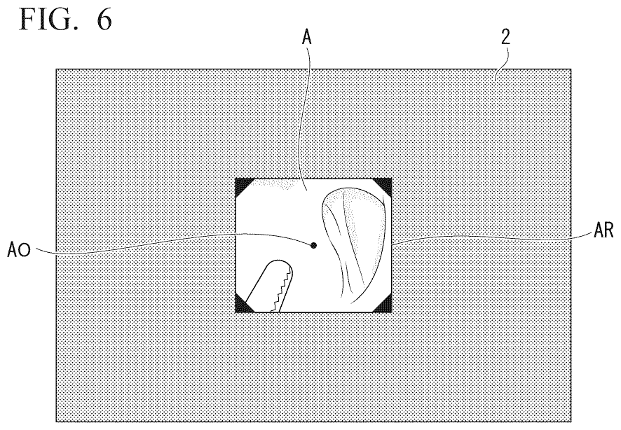

FIG. 6 is a diagram illustrating a 3D image A updated in correspondence with the new observation position P illustrated in FIG. 5. As the new observation position P illustrated in FIG. 5 becomes closer to the 3D monitor 2 than the observation position P illustrated in FIG. 2, the shorter the observation distance D becomes. For this reason, as illustrated in FIG. 6, the display size (the size of the display area AR) of the 3D image A that is determined by the display control device 4 becomes small. The display area AR is updated such that the observation position P is included in the 3D observable area R even when an observer S approaches the 3D monitor 2. As a result, even when approaching the 3D monitor 2, the observer S can observe an appropriate 3D image.

### <Parallel Movement Operation>

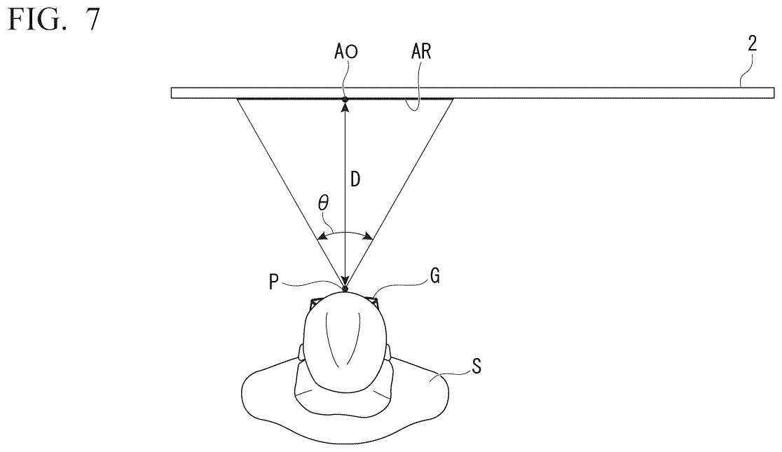

When an observer S moves parallel to a position of the left side illustrated in FIG. 7 from the position in FIG. 5, the display control device 4 determines at least one of the display size and the display position of a 3D image A displayed on the 3D monitor 2 on the basis of a new observation position P of the observer S detected by the detection device 3.

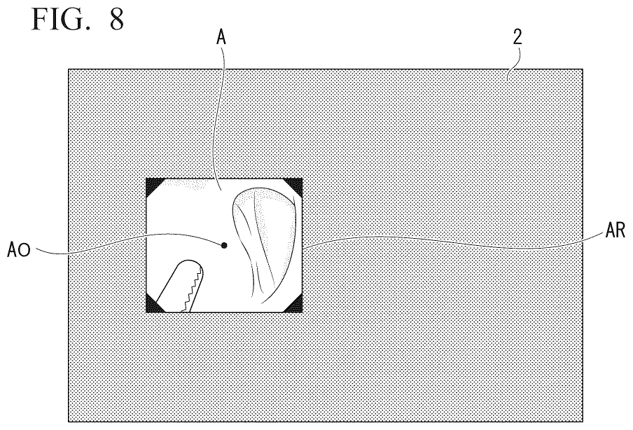

FIG. 8 is a diagram illustrating a 3D image A that has been updated in correspondence with a new observation position P illustrated in FIG. 7. When compared with the observation position P illustrated in FIG. 5, the new observation position P illustrated in FIG. 7 is located at a different position but has almost the same observation distance D. For this reason, as illustrated in FIG. 8, although the display size (the size of the display area AR) of the 3D image A determined by the display control device 4 is not changed, the center AO of the display area AR of the 3D image A is changed to the left side. The display area AR is updated such that the observation position P is included in the 3D observable area R even when the observer S moves parallel with respect to the 3D monitor 2. As a result, the observer S can observe an appropriate 3D image even when moving parallel with respect to the 3D monitor 2.

### <Crouching Operation>

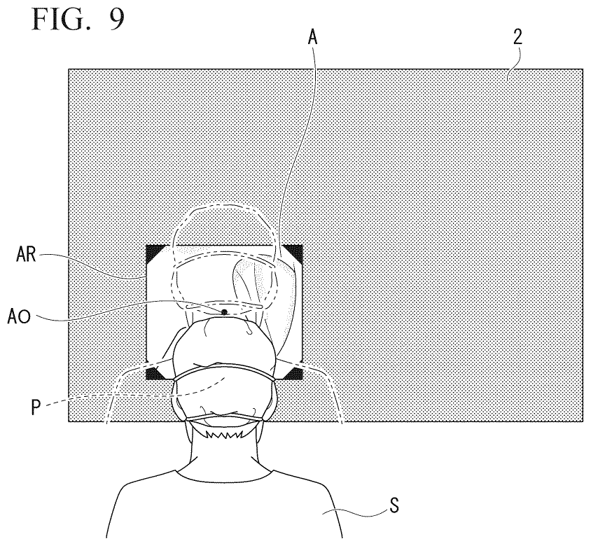

When an observer S crouches as illustrated in FIG. 9, the display control device 4 determines at least one of the display size and the display position of a 3D image A displayed on the 3D monitor 2 on the basis of a new observation position P of the observer S detected by the detection device 3.

Compared with the observation position P illustrated in FIG. 7, the new observation position P illustrated in FIG. 9 is located at a different position but has an almost same observation distance D. For this reason, as illustrated in FIG. 9, although the display size (the size of the display area AR) of a 3D image A determined by the display control device 4 is not changed, the center AO of the display area AR of the 3D image A is changed to the lower side. The display area AR is updated such that the observation position P is included in the 3D observable area R even when the observer S crouches with respect to the 3D monitor 2. As a result, an observer S can observe an appropriate 3D image even when crouching with respect to the 3D monitor 2.

### <Tracking Limit>

When an observer S moves parallel or crouches, there are cases in which at least a part of the display area AR of a 3D image A calculated on the basis of a new observation position P is outside a displayable area of the 3D monitor 2. In such cases, the display control device 4 changes the display area AR of the 3D image A to the inside of the displayable area of the 3D monitor 2. For example, the display control device 4 sets a position on the inner side of the intersection of the perpendicular line from the observation position P to the 3D monitor 2 and the 3D monitor 2 as the center AO of the display area AR of the 3D image A. As a result, the entire 3D image A is displayed on the 3D monitor 2, and the observer S can observe the entire 3D image A.

### <Plurality of Observers>

When there are two observers S observing a 3D image A displayed on the 3D monitor 2, the display control device 4 generates and displays a 3D image A for each of the observers S. The two observers S are assumed to be a first observer S1 and a second observer S2.

The display control device 4 determines the center AO1 of a display area AR1 of a 3D image A displayed on the 3D monitor 2 and the display size (the size of the display area AR1) of the 3D image A on the basis of an observation position P1 of the first observer S1 detected by the detection device 3.

The display control device 4 determines the center AO2 of a display area AR2 of a 3D image A displayed on the 3D monitor 2 and the display size (the size of the display area AR2) of the 3D image A on the basis of an observation position P2 of the second observer S2 detected by the detection device 3.

Also, in a case in which there are a plurality of observers S, the 3D observable area R is updated for each observation position P. As a result, each of the observers S can observe an appropriate 3D image. This similarly applies also to a case in which there are three or more observers S.

### <Intermittent Control: Distance>

The display control device 4 may determine at least one of the display size and the display position of a 3D image A displayed on the 3D monitor 2 on the basis of an observation area PR to which the observation position P detected by the detection device 3 belongs. As illustrated in FIG. 2, the observation area PR has a predetermined range. In a case in which the observation position P is disposed in the same observation area PR, the display control device 4 does not update the display size and the display position of the 3D image A. As a result, the display size and the display position of the 3D image A can be prevented from being excessively changed to reduce visibility.

### <Intermittent Control: Time>

The display control device 4 may determine at least one of the display size and the display position of a 3D image A displayed on the 3D monitor 2 on the basis of an observation position P detected at each predetermined time interval by the detection device 3. As a result, the display size and the display position of the 3D image A can be prevented from being excessively changed to reduce visibility.

According to the endoscope system 100 of this embodiment, an appropriate 3D image can be displayed to an observer without performing change of the position of an observer with respect to the 3D monitor and significant correction of the image.

As above, although the first embodiment of the present invention has been described in detail with reference to the drawings, a specific configuration is not limited to this embodiment, and design changes and the like in a range not departing from the concept of the present invention are included therein. In addition, the constituent elements represented in the embodiment and the modified examples described above can be appropriately combined and configured.

### Modified Example 1

In the embodiment described above, the display control device 4 generates a stereo image from an image capture signal acquired from the endoscope 1 as a 3D image A. However, the 3D image generated by the display control device 4 is not limited to a stereo image.

### Modified Example 2

In the embodiment described above, the detection device 3 is a position sensor installed in the 3D monitor 2. However, the form of the detection device 3 is not limited thereto. For example, the detection device may be an imaging unit (an imaging device) 14 of the endoscope 1. An observer S holds the imaging unit 14 in his or her hand before using the endoscope 1 and images a reference object such as a 3D monitor 2 or the like. The display control device 4 may calculate an observation position P of the observer S with respect to the 3D monitor 2 on the basis of an image acquired by imaging the reference object.

In each embodiment, the present invention may be realized by recording a program in a computer-readable recording medium and causing a computer system to read and execute the program recorded in this recording medium. The “computer system” is assumed to include an OS and hardware such as peripherals. Furthermore, the “computer-readable recording medium” represents a portable medium such as a flexible disk, a magneto-optical disk, a ROM, or a CD-ROM or a storage device such as a hard disk built into the computer system. In addition, the “computer-readable recording medium” may include a medium dynamically storing the program for a short time such as a communication line in a case in which the program is transmitted via a network such as the Internet or a communication line such as a telephone line and a medium storing the program for a predetermined time such as a volatile memory inside a computer system serving as a server or a client in the case. In addition, the program described above may be used for realizing some of the functions described above and may realize the functions described above in combination with a program that has already been recorded in the computer system.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An image display system comprising:
a monitor configured to be able to display a 3D image;
a detection device configured to detect a position of an observer or a position of monitor observation glasses with respect to the monitor as an observation position; and
a display control device configured to determine a display size of the 3D image to be displayed on the monitor such that the observation position is included in a 3D observable area of the monitor on the basis of the observation position detected by the detection device.

2. The image display system according to claim 1, wherein the display control device determines display size of the 3D image to be displayed on the monitor such that the observation position is disposed in a 3D observable area of the monitor on the basis of viewing angle of the monitor and viewing distance between the monitor and the observation position.

3. The image display system according to claim 1, wherein the display control device determines the display size of the 3D image to be displayed on the monitor on the basis of an observation distance from the monitor to the observation position.

4. The image display system according to claim 1, wherein the detection device is disposed on the monitor and is a sensor detecting the position of the observer or the position of the monitor observation glasses with respect to the monitor.

5. The image display system according to claim 1, further comprising an imaging device used by the observer,
wherein the detection device detects the observation position on the basis of an image acquired by the imaging device imaging the monitor.

6. The image display system according to claim 5, wherein the imaging device is a part of an endoscope that is able to capture the 3D image.

7. The image display system according to claim 1, wherein, when at least a part of an area displaying the 3D image determined on the basis of the observation position is outside a displayable area of the monitor, the display control device changes the area displaying the 3D image to inside the displayable area of the monitor.

8. The image display system according to claim 1, wherein the display control device determines at least one of the display size and the display position of the 3D image to be displayed on the monitor on the basis of an observation area to which the observation position detected by the detection device belongs.

9. The image display system according to claim 1, wherein the display control device determines at least one of the display size and the display position of the 3D image to be displayed on the monitor on the basis of the observation position detected by the detection device at each predetermined time interval.

10. The image display system according to claim 1,
wherein the number of the observation positions is two or more, and
wherein the 3D image is independently displayed for each of the observation positions.

11. A display control device that determines at least one of a display size and a display position of a 3D image to be displayed on a monitor on the basis of an observation position that is a position of an observer or a position of monitor observation glasses with respect to a monitor that is able to display the 3D image.

12. The display control device according to claim 11, wherein at least one of the display size and the display position of the 3D image to be displayed on the monitor is determined such that the observation position is disposed in a 3D observable area of the monitor.

13. The display control device according to claim 11, wherein the display size of the 3D image to be displayed on the monitor is determined on the basis of an observation distance from the monitor to the observation position.

14. The display control device according to claim 11, wherein, when at least a part of an area displaying the 3D image determined on the basis of the observation position is outside a displayable area of the monitor, the area displaying the 3D image is changed to inside the displayable area of the monitor.

15. The display control device according to claim 11, wherein at least one of the display size and the display position of the 3D image to be displayed on the monitor is determined on the basis of an observation area to which the observation position belongs.

16. The display control device according to claim 11, wherein at least one of the display size and the display position of the 3D image to be displayed on the monitor is determined on the basis of the observation position detected at each predetermined time interval.

17. The display control device according to claim 11,
wherein the number of the observation positions is two or more, and
wherein the 3D image is caused to be independently displayed on the monitor for each of the observation positions.

18. An image display method comprising:
a process of detecting a position of an observer or a position of monitor observation glasses with respect to a monitor as an observation position; and
a process of determining at least one of a display size and a display position of a 3D image to be displayed on the monitor on the basis of the detected observation position.

19. The image display method according to claim 18, wherein at least one of the display size and the display position of the 3D image to be displayed on the monitor is determined such that the observation position is disposed in a 3D observable area of the monitor.

20. The image display method according to claim 18, wherein the display size of the 3D image to be displayed on the monitor is determined on the basis of an observation distance from the monitor to the observation position.
